(19) United States
(12) Patent Application Publication
(10) Pub. No.: US 2010/0049045 A1
(43) Pub. Date: Feb. 25, 2010

(54) ULTRASONIC DIAGNOSTIC APPARATUS

(76) Inventors: Kiyoshi MATSUMURA, Tokyo (JP); Koji YANAGIHARA, Tokyo (JP)

Correspondence Address: PATRICK W. RASCHE (20459);ARMSTRONG TEASDALE LLP, ONE METROPOLITAN SQUARE, SUITE 2600, ST. LOUIS, MO, 63102-2740, US

(21) Appl. No.: 12/546,218

(22) Filed: Aug. 24, 2009

(30) Foreign Application Priority Data
Aug. 25, 2008 (JP) 2008-214980

(51) Int. Cl. A61B 8/14

(52) U.S. Cl. 600/437

(57) ABSTRACT

An ultrasonic diagnostic apparatus includes an ultrasonic diagnostic apparatus body adapted to generate an ultrasonic image on the basis of an echo signal obtained by transmission of an ultrasonic wave, and a display unit including a display section for the display of the ultrasonic image generated by the ultrasonic diagnostic apparatus body. The display section is supported by a support member installed on a floor surface. The display unit can be attached to and detached from the ultrasonic diagnostic apparatus body.

### CROSS REFERENCE TO RELATED APPLICATIONS

This application claims the benefit of Japanese Patent Application No. 2008-214980 filed Aug. 25, 2008, which is hereby incorporated by reference in its entirety.

### BACKGROUND OF THE INVENTION

The subject matter described herein relates to an ultrasonic diagnostic apparatus that generates an ultrasonic image on the basis of an echo signal obtained by transmission of an ultrasonic wave.

A conventional ultrasonic diagnostic apparatus is an integral combination of an operation section for an operator to input instructions, a processing section for generating an ultrasonic image on the basis of an echo signal obtained by operating the operating section to transmit an ultrasonic wave, and a display section for displaying the ultrasonic image generated by the processing section (see, for example, Japanese Unexamined Patent Publication No. 2003-339708).

In order to let the operation section be placed at a position within reach from an operator, the conventional ultrasonic diagnostic apparatus is placed on the operator's side with respect to a bed in which a subject lies. Therefore, at a bedside position and near the ultrasonic diagnostic apparatus the operator applies a probe to the subject lying in bed and performs scanning while looking at the ultrasonic image displayed on the display section. Thus, the operator performs scanning in an unnatural posture. Accordingly, it is desirable for only the display section to be movable so that the operator can see the ultrasonic image easily according to an inspection region. In view of this point the present inventor has made a study about separating the display section from the processing section and the operating section.

However, there sometimes is a case where the ultrasonic diagnostic apparatus is moved to another inspection room. If the display section is separated from the processing section and the operating section, it is necessary to move them separately, which is troublesome.

### BRIEF DESCRIPTION OF THE INVENTION

In a first aspect of the invention there is provided an ultrasonic diagnostic apparatus including an ultrasonic diagnostic apparatus body adapted to generate an ultrasonic image on the basis of an echo signal obtained by transmission of an ultrasonic wave and a display unit including a display section for the display of the ultrasonic image generated by the ultrasonic diagnostic apparatus body, the display section being supported by a support member installed on a floor surface, wherein the display unit can be attached to and detached from the ultrasonic diagnostic apparatus body.

In a second aspect of the invention there is provided, in combination with the first aspect, an ultrasonic diagnostic apparatus wherein the support member is provided with casters.

In a third aspect of the invention there is provided, in combination with the first or the second aspect, an ultrasonic diagnostic apparatus wherein the ultrasonic diagnostic apparatus body is provided with casters.

In a fourth aspect of the invention there is provided, in combination with any of the first to third aspects, an ultrasonic diagnostic apparatus wherein the support member is provided with a lift portion for raising and lowering the display section.

In a fifth aspect of the invention there is provided, in combination with any of the first to fourth aspects, an ultrasonic diagnostic apparatus wherein the ultrasonic diagnostic apparatus body and the display unit are connected together electrically through a cable.

In a sixth aspect of the invention there is provided, in combination with the fifth aspect, an ultrasonic diagnostic apparatus wherein a cable receptacle is provided in either the ultrasonic diagnostic apparatus body or the display unit.

In a seventh aspect of the invention there is provided, in combination with the sixth aspect, an ultrasonic diagnostic apparatus wherein the cable receptacle receives the cable therein by winding up the cable.

In an eighth aspect of the invention there is provided, in combination with the seventh aspect, an ultrasonic diagnostic apparatus wherein the cable receptacle has a button for starting the cable winding-up operation.

In a ninth aspect of the invention there is provided, in combination with any of the first to eighth aspects, an ultrasonic diagnostic apparatus wherein the support member has a handle portion.

In a tenth aspect of the invention there is provided, in combination with any of the first to fourth aspects, an ultrasonic diagnostic apparatus wherein the ultrasonic diagnostic apparatus body and the display unit have each a radio communication section for mutual radio communication.

In an eleventh aspect of the invention there is provided, in combination with any of the first to tenth aspects, an ultrasonic diagnostic apparatus wherein the ultrasonic diagnostic apparatus body has a processing section for processing an echo signal obtained by transmission of an ultrasonic wave and thereby generating an ultrasonic image.

In twelfth aspect of the invention there is provided, in combination with any of the first to eleventh aspects, an ultrasonic diagnostic apparatus wherein an ultrasonic probe for transmission and reception of an ultrasonic wave is connected to the processing section.

In a thirteenth aspect of the invention there is provided, in combination with the twelfth aspect, an ultrasonic diagnostic apparatus wherein the ultrasonic diagnostic apparatus body has a probe holder for holding the ultrasonic probe.

In a fourteenth aspect of the invention there is provided, in combination with any of the first to thirteenth aspects, an ultrasonic diagnostic apparatus wherein the ultrasonic diagnostic apparatus body has an operating section for an operator to input instructions.

In a fifteenth aspect of the invention there is provided, in combination with any of the first to fourteenth aspects, an ultrasonic diagnostic apparatus wherein the display unit is fitted in the ultrasonic diagnostic apparatus body detachably.

In a sixteenth aspect of the invention there is provided, in combination with the fourteenth or the fifteenth aspect, an ultrasonic diagnostic apparatus wherein the display section is an LDC.

In a seventeenth aspect of the invention there is provided, in combination with any of the first to sixteenth aspects, an ultrasonic diagnostic apparatus wherein the ultrasonic diagnostic apparatus body has a height adjusting section for adjusting the height of the operating section from an installation surface on which the ultrasonic diagnostic apparatus body is installed.

According to the first aspect, the display unit can be attached to and detached from the ultrasonic diagnostic apparatus body, so by detaching the display unit from the ultrasonic diagnostic apparatus body it becomes possible to place the display unit at a position where the operator is easy to see the ultrasonic image. Consequently, the operator can perform scanning in a natural posture. On the other hand, when the ultrasonic diagnostic apparatus is to be moved, it can be moved easily in the attached state of the display unit to the ultrasonic diagnostic apparatus body.

According to the second aspect, the support member can be moved easily because it is provided with casters.

According to the third aspect, the ultrasonic diagnostic apparatus can be moved easily because the ultrasonic diagnostic apparatus body is provided with casters.

According to the fourth aspect, since the support member is provided with a lift portion for raising and lowering the display section, a vertical position of the display section can be changed. Consequently, the display section can be positioned at a height at which the operator is easy to see the display section.

According to the fifth aspect, data of the ultrasonic image generated in the ultrasonic diagnostic apparatus body is transmitted to the display unit through the cable.

According to the sixth aspect, the cable can be prevented from becoming an obstacle because it can be received in the cable receptacle.

According to the seventh aspect, the cable is wound up into the cable receptacle.

According to the eighth aspect, the cable winding-up operation is started by pushing the button.

According to the ninth aspect, the operator can move the support member while grasping the handle portion.

According to the tenth aspect, the cable for connection between the ultrasonic diagnostic apparatus body and the display unit can be omitted.

According to the eleventh aspect, an ultrasonic image is generated in the processing section provided in the ultrasonic diagnostic apparatus body.

According to the twelfth aspect, the transmission and reception of an ultrasonic wave are performed in the ultrasonic probe connected to the processing section.

According to the thirteenth aspect, the ultrasonic probe can be held by the probe holder.

According to the fourteenth aspect, the operator inputs instructions in the operating section provided in the ultrasonic diagnostic apparatus body.

According to the fifteenth aspect, the display unit is fitted in the ultrasonic diagnostic apparatus body detachably.

According to the sixteenth aspect, the display section can be made thin because it is an LCD.

According to the seventeenth aspect, the height of the operating section from the installation surface can be adjusted so that the operator is easy is operate the operating section.

Further objects and advantages of the invention will be apparent from the following description of the preferred embodiments of the invention as illustrated in the accompanying drawings.

### BRIEF DESCRIPTION OF THE DRAWINGS

FIG. 1 is a perspective view of an ultrasonic diagnostic apparatus according to an embodiment of the invention.

FIG. 2 is also a perspective view of the ultrasonic diagnostic apparatus according to an embodiment of the invention.

FIG. 3 is a side view showing an integrally combined state of both an ultrasonic diagnostic apparatus body and a display unit.

FIG. 4 is a plan view showing a usage example of the ultrasonic diagnostic apparatus.

FIG. 5 is a perspective view showing a modification of the embodiment.

### DETAILED DESCRIPTION OF THE INVENTION

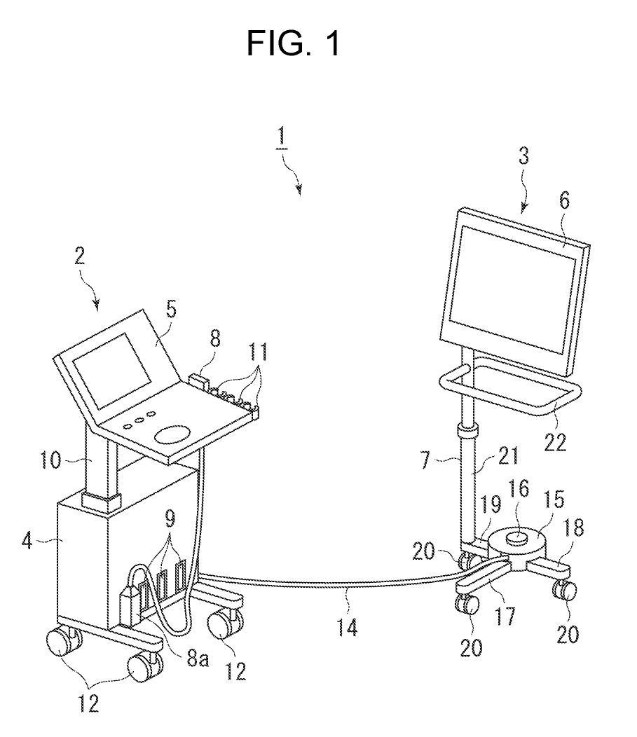

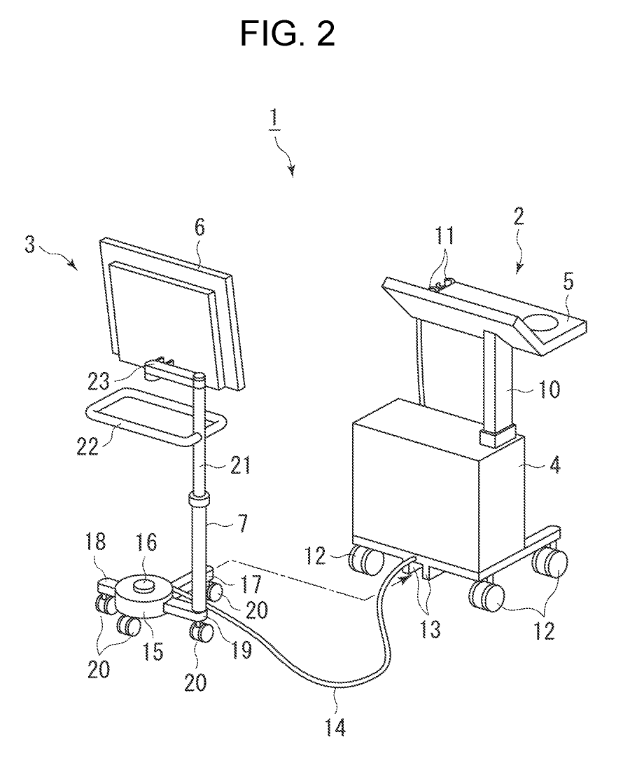

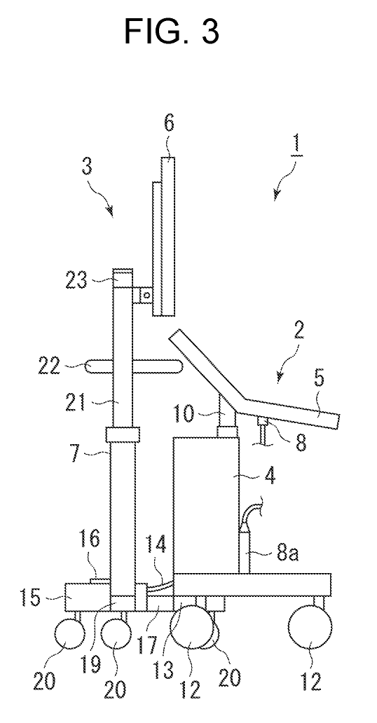

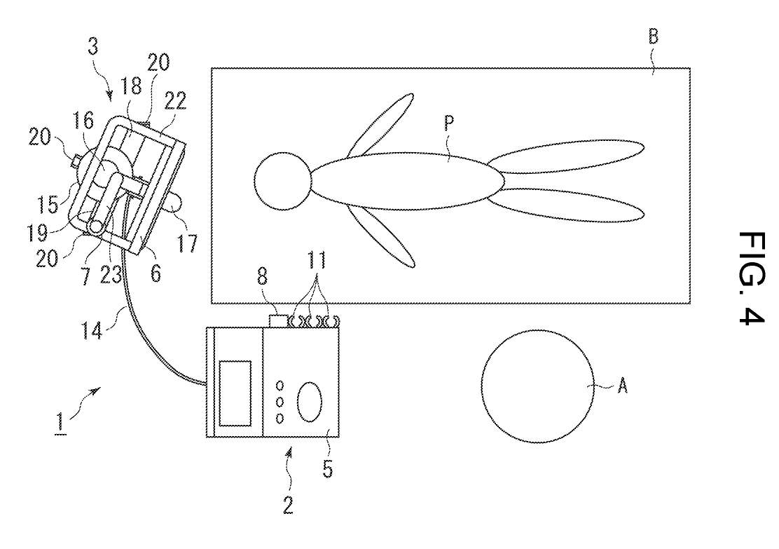

Embodiments of the invention will be described below in detail with reference to the drawings. FIGS. 1 and 2 are perspective views of an ultrasonic diagnostic apparatus according to an embodiment of the invention, FIG. 3 is a side view showing an ultrasonic diagnostic apparatus body and a display unit both combined integrally with each other, and FIG. 4 is a plan view showing a usage example of the ultrasonic diagnostic apparatus embodying the invention.

The ultrasonic diagnostic apparatus, indicated at 1, includes an ultrasonic diagnostic apparatus body 2 and a display unit 3. The ultrasonic diagnostic apparatus body 2 includes a processing section 4 for processing an echo signal obtained by transmission of an ultrasonic wave and thereby generating an ultrasonic image and an operating section 5 for an operator to input instructions. The display unit 3 includes a display section 6 for displaying the ultrasonic image generated in the ultrasonic diagnostic apparatus body 2 and a support member 7 installed on a floor surface to support the display section 6.

An ultrasonic probe 8 for the transmission and reception of an ultrasonic wave is connected to the processing section 4. The processing section 4 is provided with probe connectors 9 to which a cable connector 8a of the ultrasonic probe 8 is connected. In accordance with instructions inputted by the operator in the operating section 5 and on the basis of an echo signal obtained by operating the ultrasonic probe 8 the processing section 4 makes control to generate an ultrasonic image and display it on the display section 6.

The operating section 5 is supported by a support post 10 which projects upward from the interior of the processing section 4. A projecting height of the support post 10 from the processing section 4 can be adjusted, whereby the height of the operating section 5 from the floor surface can be adjusted.

The support post 10 is an example of the height adjusting section defined in the invention.

The operating section 5 includes a pointing device such as a mouse or a tracking ball and a keyboard (neither shown). In a side portion of the operating section 5 there are provided four probe holders 11 for holding the ultrasonic probe 8.

Casters 12 are provided on the underside of the ultrasonic diagnostic apparatus body 2. A pair of engaging pieces 13 (FIG. 2) for fitting therein of the display unit 3 are also formed on the underside of the ultrasonic diagnostic apparatus body 2. A long leg 17 to be described later of the display unit 3 is fitted between the pair of engaging pieces 13 detachably.

The ultrasonic diagnostic apparatus body 2 and the display unit 3 are connected together electrically through a cable 14. The support member 7 of the display unit 3 is provided with a cable receptacle 15 for receiving the cable 14 therein. The cable receptacle 15 receives the cable 14 therein by winding up the cable. The cable receptacle 15 has a button 16 to start winding-up of the cable 14.

The cable receptacle 15 is provided with a long leg 17 and short legs 18 and 19 orthogonal to the long leg 17. The long leg 17, the short legs 18, 19 and the cable receptacle 15 are each provided with one caster 20.

A support post 21 is erected on the short leg 19 and it is provided with a handle portion 22. The display section 6 is attached to an upper end of the support post 21 through an arm 23. The support post 21 can extend and retract. The display section 6 can be moved up and down by extending and retracting the support post 21. The support post 21 is an example of the lift portion defined in the invention.

The arm 23 is pivotable horizontally at its mounted portion on the support post 21. Likewise, the display section 6 is pivotable horizontally at its mounted portion to the arm 23. The display section 6 is also pivotable vertically. The display section 6 is constituted by an LCD (Liquid Crystal Display).

In case of conducting an inspection with use of the ultrasonic diagnostic apparatus 1 of this embodiment, the ultrasonic diagnostic apparatus body 2 and the display unit 3 are placed at a position where the operator is easy to conduct the inspection. For example, for inspection of the abdomen, as shown in FIG. 4, the display unit 3 is detached from the ultrasonic diagnostic apparatus body 2, then the ultrasonic diagnostic apparatus body 2 is placed on an operator's chair A side with respect to a bed B so that the operator is easy to operate the operating section 5, further, the display unit 3 is placed on the head side of a subject P lying in the bed B so that the operator is easy to look at the display section 6. At this time, by moving the display unit 3 while grasping the handle portion 22, the operator can move the display unit 3 easily up to a desired position.

For inspecting lower limbs, though not specially shown, the display unit 3 is placed on the foot side of the subject P lying on the bed B. Thus, according to each inspection region, the operator places the display unit 3 at a position where the operator is easy to see the display section 6.

After the ultrasonic diagnostic apparatus body 2 and the display unit 3 have thus been placed at respective appropriate positions, an extra portion of the cable 14 is received into the cable receptacle 15, whereby the cable can be prevented from becoming an obstacle.

Before conducting each inspection, the operator adjusts the height of the display section 6 so that the operator is easy to see the display section. Further, the height of the operating section 5 is adjusted to a height that permits easy operation of the operator.

On the other hand, when moving the ultrasonic diagnostic apparatus 1 from a certain inspection room to another inspection room, the long leg 17 is fitted in between the pair of engaging pieces 13 to combine the ultrasonic diagnostic apparatus body 2 and the display unit 3 integrally with each other, as shown in FIG. 3. The ultrasonic diagnostic apparatus body 2 and the display 2 are moved in such an integrally combined state to another inspection room. If an inspection is to be conducted again after the movement, the display unit 3 is detached from the ultrasonic diagnostic apparatus body 2 and is placed at a position where the operator is easy to see an ultrasonic image.

When combining the ultrasonic diagnostic apparatus body 2 and the display unit 3 integrally with each other, the cable 14 is wound up into the cable receptacle 15 by pushing the button 16 of the cable receptacle 15, whereby the cable 14 can be prevented from becoming an obstacle during the movement.

According to the ultrasonic diagnostic apparatus 1 of this embodiment, the display unit 3 can be attached to and detached from the ultrasonic diagnostic apparatus body 2, so by detaching the display unit 3 from the ultrasonic diagnostic apparatus body 2, the display unit 3 can be placed at a position where the operator is easy to see an ultrasonic image. Consequently, the operator can perform scanning in a natural posture. On the other hand, in case of moving the ultrasonic diagnostic apparatus 1, the display unit 3 can be moved in its mounted state to the ultrasonic diagnostic apparatus body 2 and thus can be moved easily.

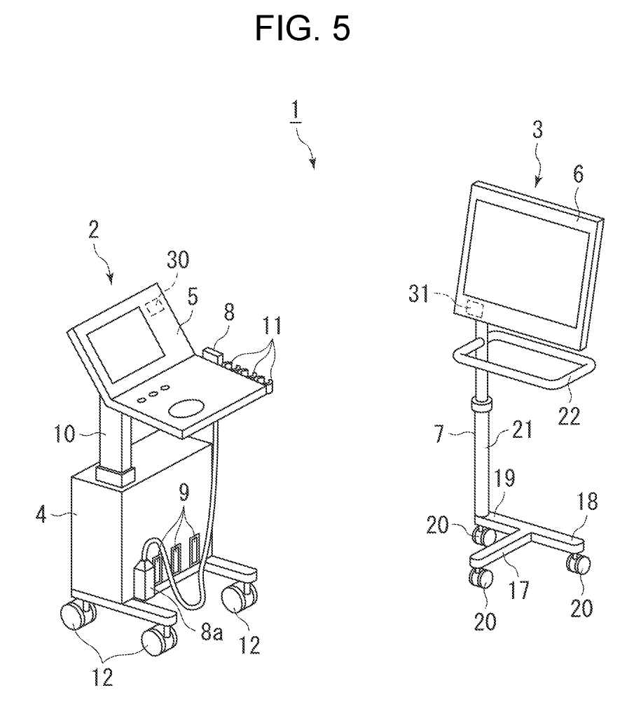

Although the invention has been described by way of the above embodiment, it goes without saying that various changes may be made within the scope not departing from the gist of the invention. For example, the ultrasonic diagnostic apparatus body 2 and the display unit 3 may be coupled together not through the cable but by radio communication. More particularly, as shown in FIG. 5, the ultrasonic diagnostic apparatus body 2 and the display unit 3 may have radio communication sections 30 and 31, respectively, for mutual radio communication. In this case, the display unit 3 does not have the cable receptacle 15 because the cable 14 for connection between the ultrasonic diagnostic apparatus body 2 and the display unit 3 is not used.

In case of using the cable 14, the cable receptacle 15 may be provided in the ultrasonic diagnostic apparatus body 2.

Many widely different embodiments of the invention may be configured without departing from the spirit and the scope of the present invention. It should be understood that the present invention is not limited to the specific embodiments described in the specification, except as defined in the appended claims.

1. An ultrasonic diagnostic apparatus comprising:
an ultrasonic diagnostic apparatus body adapted to generate an ultrasonic image based on an echo signal obtained by transmission of an ultrasonic wave; and
a display unit comprising a display section configured to display the ultrasonic image the display section supported by a support member installed on a floor surface, wherein the display unit is further configured to be selectively attached to and detached from the ultrasonic diagnostic apparatus body.

2. An ultrasonic diagnostic apparatus according to claim 1, wherein the support member comprises a first plurality of casters.

3. An ultrasonic diagnostic apparatus according to claim 2, wherein the ultrasonic diagnostic apparatus body comprises a second plurality of casters.

4. An ultrasonic diagnostic apparatus according to claim 3, wherein the support member comprises a lift portion configured to selectively raise and lower the display section.

5. An ultrasonic diagnostic apparatus according to claim 1, wherein the ultrasonic diagnostic apparatus body and the display unit are connected together electrically through a cable.

6. An ultrasonic diagnostic apparatus according to claim 5, wherein at least one of the ultrasonic diagnostic apparatus body and the display unit comprises a cable receptacle.

7. An ultrasonic diagnostic apparatus according to claim 6, wherein the cable receptacle receives the cable therein by winding up the cable.

8. An ultrasonic diagnostic apparatus according to claim 7, wherein the cable receptacle comprises a button configured to start a cable winding-up operation.

9. An ultrasonic diagnostic apparatus according to claim 1, wherein the support member comprises a handle portion.

10. An ultrasonic diagnostic apparatus according to claim 1, wherein the ultrasonic diagnostic apparatus body and the display unit each comprises a radio communication section for mutual radio communication.

11. An ultrasonic diagnostic apparatus according to claim 1, wherein the ultrasonic diagnostic apparatus body comprises a processing section configured to process the echo signal and to generate the ultrasonic image.

12. An ultrasonic diagnostic apparatus according to claim 1, further comprising an ultrasonic probe configured to transmit and receive the ultrasonic wave, the ultrasonic probe connected to the processing section.

13. An ultrasonic diagnostic apparatus according to claim 12, wherein the ultrasonic diagnostic apparatus body comprises a probe holder configured to hold the ultrasonic probe.

14. An ultrasonic diagnostic apparatus according to claim 1, wherein the ultrasonic diagnostic apparatus body comprises an operating section configured to receive input instructions from an operator.

15. An ultrasonic diagnostic apparatus according to claim 1, wherein the display unit is fitted in the ultrasonic diagnostic apparatus body detachably.

16. An ultrasonic diagnostic apparatus according to claim 5, wherein the display unit is fitted in the ultrasonic diagnostic apparatus body detachably.

17. An ultrasonic diagnostic apparatus according to claim 15, wherein the display section comprises an LCD.

18. An ultrasonic diagnostic apparatus according to claim 1, wherein the ultrasonic diagnostic apparatus body comprises a height adjusting section configured to adjust a height of the operating section from an installation surface on which the ultrasonic diagnostic apparatus body is installed.

19. An ultrasonic diagnostic apparatus according to claim 5, wherein the ultrasonic diagnostic apparatus body comprises a height adjusting section configured to adjust a height of the operating section from an installation surface on which the ultrasonic diagnostic apparatus body is installed.

20. An ultrasonic diagnostic apparatus according to claim 15, wherein the ultrasonic diagnostic apparatus body comprises a height adjusting section configured to adjust a height of the operating section from an installation surface on which the ultrasonic diagnostic apparatus body is installed.
